(19) United States
(12) Patent Application Publication
(10) Pub. No.: US 2003/0114731 A1
(43) Pub. Date: Jun. 19, 2003

(54) Magnetic positioning system for trocarless laparoscopic instruments

(76) Inventors: Jeffrey A. Cadeddu, Dallas, TX (US); Linda A. Baker, Southlake, TX (US); Richard Bergs, Rowlett, TX (US)

Correspondence Address: Edwin S. Flores, Gardere Wynne Sewell LLP, 3000 Thanksgiving Tower, 1601 Elm Street, Suite 3000, Dallas, TX, 75201-4767, US

(21) Appl. No.: 10/024,636

(22) Filed: Dec. 14, 2001

(51) Int. Cl. A61B 19/00; A61B 1/01

(52) U.S. Cl. 600/114; 606/130; 600/145

(57) ABSTRACT

The present invention relates to methods and apparatuses for performing surgery, and in particular to devices employing magnetic fields to position and orient medical instruments inside a human body. To provide for greater flexibility of endoscopic viewing and instrument usage and to reduce morbidity, the inventors have developed of a novel laparoscopic system that allows for intra-abdominal movement of an endoscopic camera and surgical instruments without additional port sites. A set of one or more magnets located external to the patient's body are used to position, orient, and/or secure instruments located internal to the patient's body.

### TECHNICAL FIELD OF THE INVENTION

The present invention relates to a method and apparatus for performing surgery, and in particular to employing magnetic fields to position and orient medical instruments inside a human body.

### BACKGROUND OF THE INVENTION

Many surgical procedures are now being performed with the use of trocars and cannulas. Originally these devices were used for making a puncture and leaving a tube to drain fluids. As technology and surgical techniques have advanced, it is now possible to insert surgical instruments through the cannulas and perform invasive procedures through openings less than half an inch in diameter. These surgical procedures previously required incisions of many inches. By minimizing the incision, the stress and loss of blood suffered by a patient is reduced and the patient's recovery time is dramatically reduced.

Surgical trocars are most commonly used in laparoscopic surgery. Prior to use of the trocar, the surgeon will usually introduce a Veress needle into the patient's abdominal cavity. The Veress needle has a stylet, which permits the introduction of gas into the abdominal cavity. After the Veress needle is properly inserted, it is connected to a gas source and the abdominal cavity is insufflated to an approximate abdominal pressure of 15 mm Hg. By insufflating the abdominal cavity, pneumoperitoneum is created separating the wall of the body cavity from the internal organs.

A trocar is then used to puncture the body cavity. The piercing tip or obturator of the trocar is inserted through the cannula or sheath and the cannula partially enters the body cavity through the incision made by the trocar. The obturator can then be removed from the cannula and an elongated endoscope or camera may be inserted through the cannula to view the body cavity, or surgical instruments may be inserted to perform ligations or other procedures.

A great deal of force is often required to cause the obturator to pierce the wall of the body cavity. When the piercing tip breaks through the cavity wall, resistance to penetration ceases and the tip may reach internal organs or blood vessels, with resultant lacerations and potentially serious injury. The creation of the pneumoperitoneum provides some free space within which the surgeon may stop the penetration of the trocar. To provide further protection, trocars have more recently been developed with spring loaded shields surrounding the piercing tip of the obturator. Once the piercing tip of the obturator has completely pierced the body cavity wall, the resistance of the tissue to the spring-loaded shield is reduced and the shield springs forward into the body cavity and covers the piercing tip. The shield thereby protects internal body organs and blood vessels from incidental contact with the piercing tip and resultant injury.

Once the cannula has been introduced into the opening in the body cavity wall, the pneumoperitoneum may be maintained by introducing gas into the abdominal cavity through the cannula. Various seals and valves have been used to allow abdominal pressure to be maintained in this fashion. Maintaining abdominal pressure is important both to allow working room in the body cavity for instruments introduced through the cannula and to provide free space for the puncturing of the body cavity wall by one or more additional trocars as may be required for some procedures.

A principal limitation of traditional laparoscopy relates to the fixed working envelope surrounding each trocar. These relatively small working envelopes often necessitate the placement of multiple ports in order to accommodate necessary changes in instrument position and to improve visibility and efficiency. The creation of additional ports is known to contribute to post-operative pain and to increase the risk of bleeding or organ damage.

### SUMMARY OF THE INVENTION

The following summary of the invention is provided to facilitate an understanding of some of the innovative features unique to the present invention, and is not intended to be a full description. A full appreciation of the various aspects of the invention can only be gained by taking the entire specification, claims, drawings and abstract as a whole.

The present invention relates to a method and apparatus for manipulation of surgical instruments within the human body. Although methods have been developed for manipulation of such instruments from outside the body, numerous limitations have been identified in connection with prior methods.

Accordingly, the present inventors recognized that the field of laparoscopic surgery needs a method and apparatus that enables a surgeon to manipulate the position and orientation of one or more instruments within a human body without the necessity for multiple trocars. To provide for greater flexibility of endoscopic viewing and instrument usage and to further reduce morbidity, the inventors have developed a novel laparoscopic system that allows for unrestricted intra-abdominal movement of an endoscopic camera and surgical instruments without additional port sites.

In the present invention, a set of one or more magnets located external to the patient's body are used to position, orient and/or secure instruments located internal to the patient's body. Certain embodiments of the present invention employ a method incorporating the steps of: generating a magnetic field in alignment with the principal magnetic axis of the magnetically-attractive element and reorienting the magnetic field in such a manner as to reorient the laparoscopic instrument to the second orientation.

In another embodiment, the method includes: attaching rigidly to the laparoscopic instrument a magnetically-attractive element having at least one principal magnetic axis, generating a magnetic field in alignment with the principal magnetic axis of the magnetically-attractive element and reorienting the magnetic field in such a manner as to reorient the laparoscopic instrument to the second orientation

The method of the present invention may include the steps of: inserting a laparoscopic instrument having light-sensitive elements and a magnetically-attractive element into the body, applying a magnetic field to the magnetically-attractive element in such a manner as to orient the laparoscopic instrument to a desired orientation, and exposing the light-sensitive elements of the laparoscopic instrument to light reflected from a desired internal feature.

Alternatively, the method of the present invention may include the steps of: applying to the magnetically-attractive element a first magnetic field aligned with a first principal axis and applying to the magnetically-attractive element a second magnetic field aligned with a second principal axis.

In another example, the present invention includes the steps of: detecting the first position of a laparoscopic instrument, applying to a magnetically-attractive element within the instrument a first magnetic field aligned with a first principal axis so as to translate the instrument from the first position to a second position, detecting the instrument orientation and applying to the magnetically-attractive element a second magnetic field so as to reorient the laparoscopic instrument to the second orientation.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The accompanying figures, in which like reference numerals refer to identical or functionally-similar elements throughout the separate views and which are incorporated in and form part of the specification, further illustrate the present invention and, together with the detailed description of the invention, serve to explain the principles of the present invention.

FIG. 1 is a side section view of a patient undergoing laparoscopic surgery showing a laparoscopic instrument being manipulated by the use of external magnets according to one embodiment of the present invention;

FIG. 2 is a side section view of a laparoscopic surgery patient showing a laparoscopic instrument being manipulated by an array of external magnets according to a second embodiment of the present invention;

FIG. 3 is a transverse section view of a laparoscopic surgery patient showing a laparoscopic instrument being manipulated by an array of external magnets according to a third embodiment of the present invention;

FIG. 4 is a transverse section view of a laparoscopic surgery patient showing a laparoscopic instrument being manipulated by an array of external magnets according to a fourth embodiment of the present invention;

FIG. 5 is a side section view of a laparoscopic surgery patient showing a laparoscopic instrument being manipulated by an array of external magnets according to a fifth embodiment of the present invention; and

FIG. 6 is a side section view of a laparoscopic surgery patient showing a laparoscopic instrument being manipulated by an array of external magnets according to a sixth embodiment of the present invention.

### DETAILED DESCRIPTION OF THE DRAWINGS

The embodiments and examples set forth herein are presented to best explain the present invention and its practical application and to thereby enable those skilled in the art to make and utilize the invention. Those skilled in the art, however, will recognize that the description and examples are presented for the purpose of illustration and example only. Other variations and modifications of the present invention will be apparent to those of skill in the art, and it is the intent of the appended claims that such variations and modifications be covered.

The description as set forth is not intended to be exhaustive or to limit the scope of the invention. Many modifications and variations are possible in light of the above teaching without departing from the spirit and scope of the following claims. It is contemplated that the use of the present invention can involve components having different characteristics. It is intended that the scope of the present invention be defined by the claims appended hereto, giving full cognizance to equivalents in all respects.

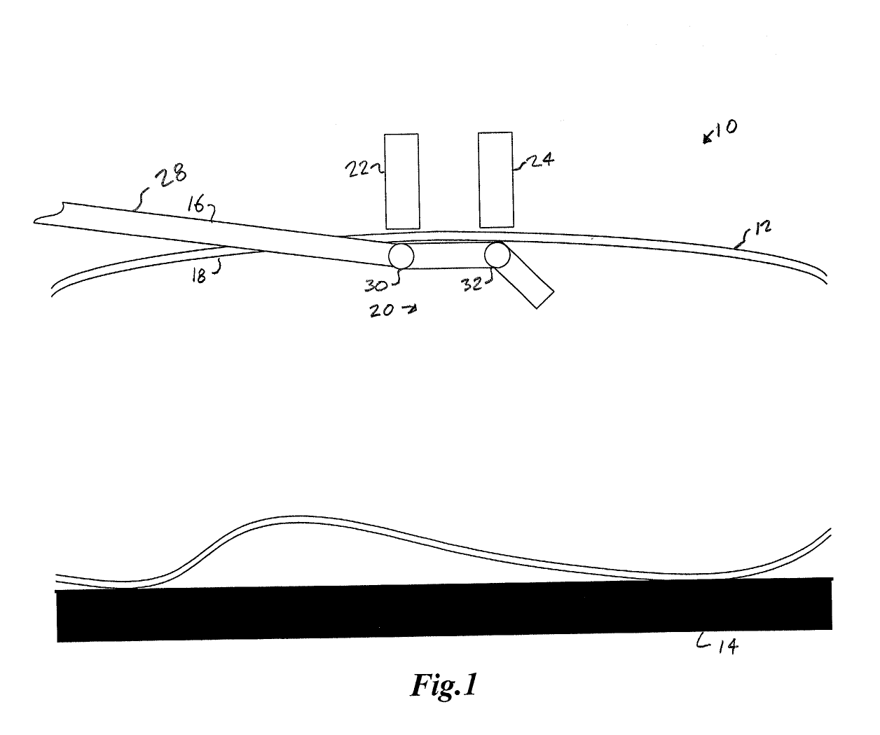

FIG. 1 is a side section view depicting a laparoscopic surgery apparatus 10 in accordance with certain embodiments of the present invention. Laparoscopic surgery apparatus 10 incorporates a surface 14 for supporting the patient 12, one or more laparoscopic instruments 16, and one or more magnetic sources, such as magnetic sources 22 and 24. In certain embodiments, laparoscopic instrument 16 may be, as an example, an endoscope. FIG. 1, laparoscopic instrument 16 is shown protruding through the outer surface 18 of the patient 12, such that at least a portion of the laparoscopic instrument 16 protrudes into an inner cavity 20 within the patient 12.

In traditional forms of laparoscopic surgery, laparoscopic instruments 16 inserted into a body cavity 20 were principally manipulated by the application of force to the portion 28 of the laparoscopic instrument 16 protruding from the patient 12. Although this method is useful for adjusting the depth of insertion of the laparoscopic instrument 16 and can provide a limited range of angular or side-to-side movement, all but minor changes in the orientation of the laparoscopic instrument 16 had to be accomplished through the creation of additional incisions in the patient 12.

Owing to the use of magnetic fields to position, orient and affix laparoscopic instrument 16 within the body cavity 20, a surgeon's control over the position and orientation of laparoscopic instrument 16 can be controlled with much greater flexibility. As can be seen in FIG. 1, the position and orientation of laparoscopic instrument 16 is controlled in part by magnetic field sources 22 and 24.

The laparoscopic instrument 16 shown in FIG. 1 is fixed in place by the manipulation, by magnetic field sources 30 and 32, of magnetic portions 30 and 32 on the laparoscopic instrument 16. In certain embodiments, magnetic field sources 30 and 32 may be permanent magnets generating a magnetic field of a constant strength. In other embodiments, magnetic field sources 30 and 32 may be electromagnets generating a field of a constant strength, a variable strength, or a varying time-dependent strength. Magnetic field sources 30 and 32 may be single magnetic sources, or may be composed of arrays of smaller sources.

Similarly, magnetically-attractive portions 30 and 32 may be ferromagnetic materials, permanent magnets, or electromagnets. In embodiments wherein magnetically-attractive portions 30 and 32 are electromagnets, the magnetically-attractive portions 30 and 32 may be selectively energized or de-energized, may be adjustable across a range, or may be subjected to a time-dependent signal such as a square or sinusoidal wave. Such functionality may be employed to provide independent positional control of two or more magnetically-attractive 30 and 32.

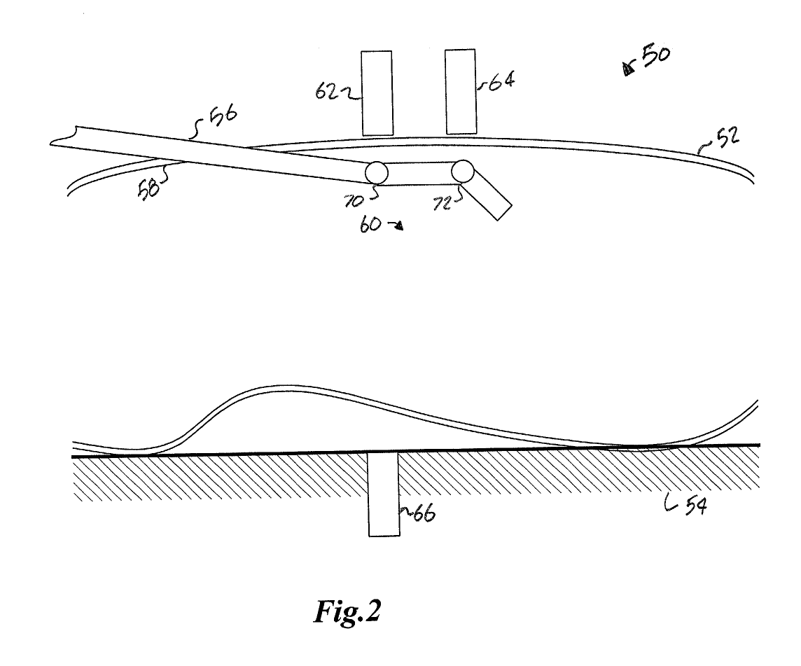

FIG. 2 is a side section view depicting a laparoscopic surgery apparatus 50 in accordance with certain embodiments of the present invention. Laparoscopic surgery apparatus 50 incorporates a surface 54 for supporting the patient 52, one or more laparoscopic instruments 56, and one or more magnetic sources, such as magnetic sources 62 and 64. FIG. 2, laparoscopic instrument 56 is shown protruding through the outer surface 58 of the patient 52, such that at least a portion of the laparoscopic instrument 56 protrudes into an inner cavity 60 within the patient 52.

Owing to the use of magnetic fields to position, orient, and affix laparoscopic instrument 56 within the body cavity 60, a surgeon's control over the position and orientation of laparoscopic instrument 56 can be controlled with much greater flexibility. As can be seen in FIG. 2, the position and orientation of laparoscopic instrument 56 is controlled in part by magnetic field sources 62, 64 and 66. The laparoscopic instrument 56 shown in FIG. 1 is fixed in place by the manipulation, by magnetic field sources 62, 64 and 66, of magnetic portions 70 and 72 on the laparoscopic instrument 56.

Manipulated appropriately, the incorporation of a third magnetic field source 66 allows the surgeon or other operator to exert an extra degree of control over the position and orientation of laparoscopic instrument 56. In certain embodiments, appropriate time-varying signals may be applied to magnetic field sources 62, 64 and 66 in such a manner that one or more nodes may be created in the magnetic field. Such nodes may be used to manipulate the magnetically-attractive portions 70 and 72 as desired.

In an alternate embodiment, magnetic field generators 62, 64 and 66 may be aligned orthogonally to one another, such that the characteristics of the fields generated by the respective magnetic field generators may be varied independently so as to provide three-dimensional positional control. Similarly, a combination of six magnetic field generators may be arranged in pairs along orthogonal principal axes for the same functionality.

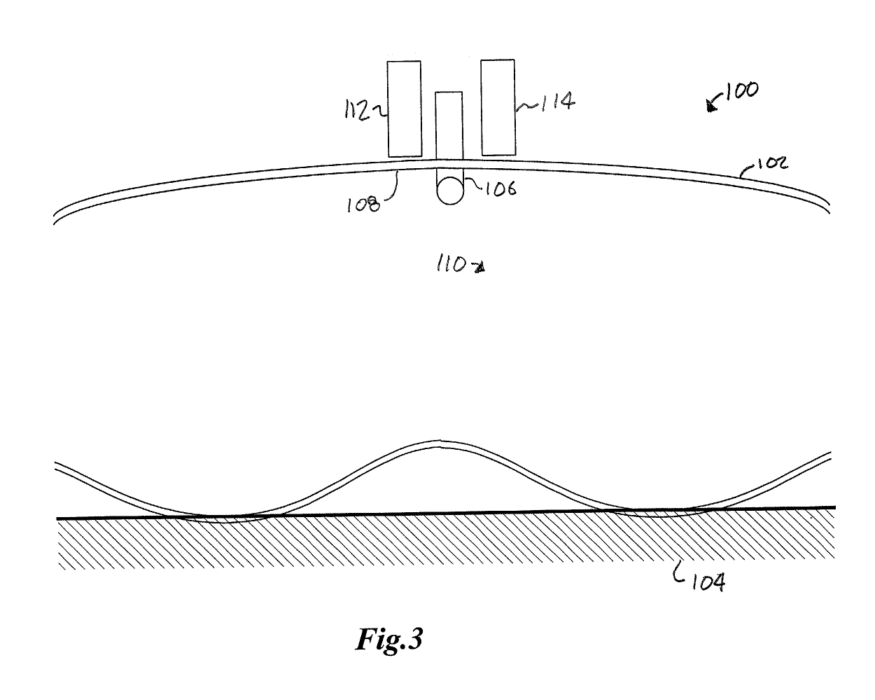

FIG. 3 is a transverse section view depicting a laparoscopic surgery apparatus 100 in accordance with certain embodiments of the present invention. Laparoscopic surgery apparatus 100 incorporates a surface 104 for supporting the patient 102, one or more laparoscopic instruments 106, and one or more magnetic sources, such as magnetic sources 112 and 114. FIG. 3, laparoscopic instrument 106 is shown protruding through the outer surface 108 of the patient 102, such that at least a portion of the laparoscopic instrument 106 protrudes into an inner cavity 110 within the patient 102.

Owing to the use of magnetic fields to position, orient, and affix laparoscopic instrument 106 within the body cavity 110, a surgeon's control over the position and orientation of laparoscopic instrument 106 may also be controlled with much greater flexibility. As can be seen in FIG. 3, the position and orientation of laparoscopic instrument 106 is controlled in part by magnetic field sources 112 and 114. The laparoscopic instrument 106 shown in FIG. 3 is fixed in place by the manipulation, by magnetic field sources 112 and 114, of magnetic portions on the laparoscopic instrument 106.

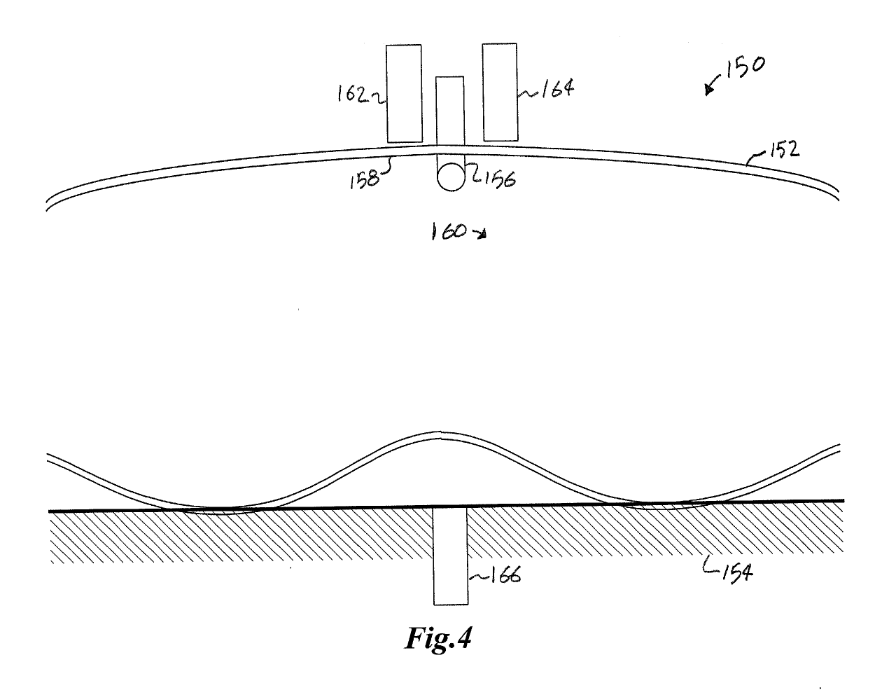

FIG. 4 is a transverse section view depicting a laparoscopic surgery apparatus 150 in accordance with certain embodiments of the present invention. Laparoscopic surgery apparatus 150 incorporates a surface 154 for supporting the patient 152, one or more laparoscopic instruments 156, and one or more magnetic sources, such as magnetic sources 162 and 164. FIG. 4, laparoscopic instrument 156 is shown protruding through the outer surface 158 of the patient 152, such that at least a portion of the laparoscopic instrument 156 protrudes into an inner cavity 110 within the patient 152.

Owing to the use of magnetic fields to position, orient, and affix laparoscopic instrument 156 within the body cavity 160, a surgeon's control over the position and orientation of laparoscopic instrument 156 can be controlled with much greater flexibility. As can be seen in FIG. 4, the position and orientation of laparoscopic instrument 156 is controlled in part by magnetic field sources 162, 164, and 166. The laparoscopic instrument 156 shown in FIG. 4 is fixed in place by the manipulation, by magnetic field sources 162, 164, and 166, of magnetic portions on the laparoscopic instrument 156.

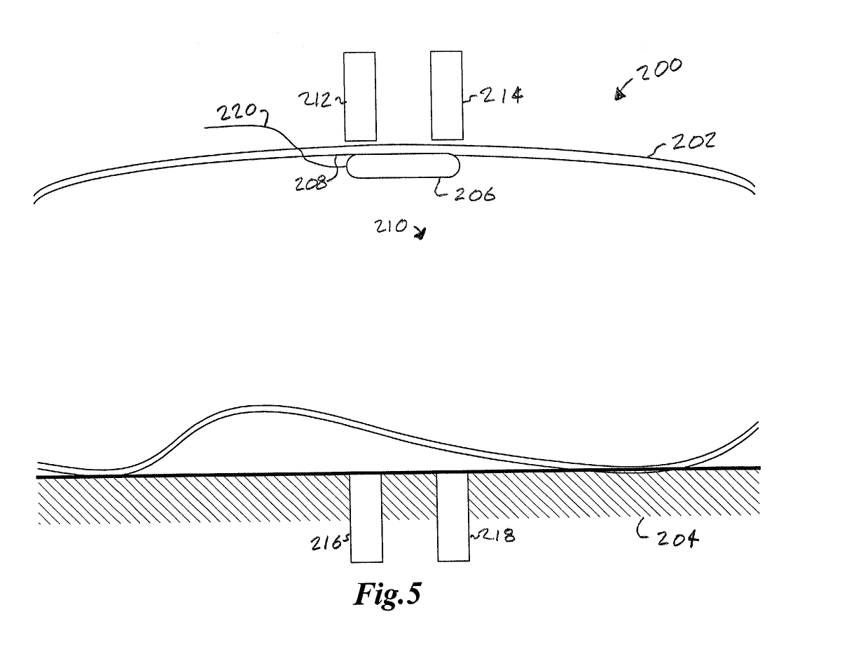

FIG. 5 is a length-wise section view depicting a laparoscopic surgery apparatus 200 in accordance with certain embodiments of the present invention. Laparoscopic surgery apparatus 200 incorporates a surface 204 for supporting the patient 202, one or more laparoscopic instruments 206, and magnetic sources 212, 214, 216 and 218. FIG. 5, laparoscopic instrument 206 is shown disposed within the outer surface 208 of the patient 202, such that the laparoscopic instrument 206 is disposed within an inner cavity 210 within the patient 202.

Owing to the use of magnetic fields to position, orient and affix laparoscopic instrument 206 within the body cavity 210, a surgeon's control over the position and orientation of laparoscopic instrument 206 can be controlled with much greater flexibility. As can be seen in FIG. 5, the position and orientation of laparoscopic instrument 206 is controlled in part by magnetic field sources 212, 214, 216 and 218. The laparoscopic instrument 206 shown in FIG. 5 is fixed in place by the manipulation, by magnetic field sources 212, 214, 216 and 218, of magnetic portions on the laparoscopic instrument 206.

In contrast to the embodiments shown in FIGS. 1-4, laparoscopic surgery apparatus 200 does not incorporate a laparoscopic instrument 206 having any rigid or structural portion remaining outside the body of the patient 202. In this embodiment, all positional and orientation control of laparoscopic instrument 206 must be effectuated through the use of magnetic field generators 212-218. In certain embodiments, a certain amount of control input may be provided through link 220. In embodiments in which laparoscopic instrument 206 incorporates an endoscope or other type of sensor, link 220 may be used to relay video or other sensory information from laparoscopic instrument 206 to the outside world.

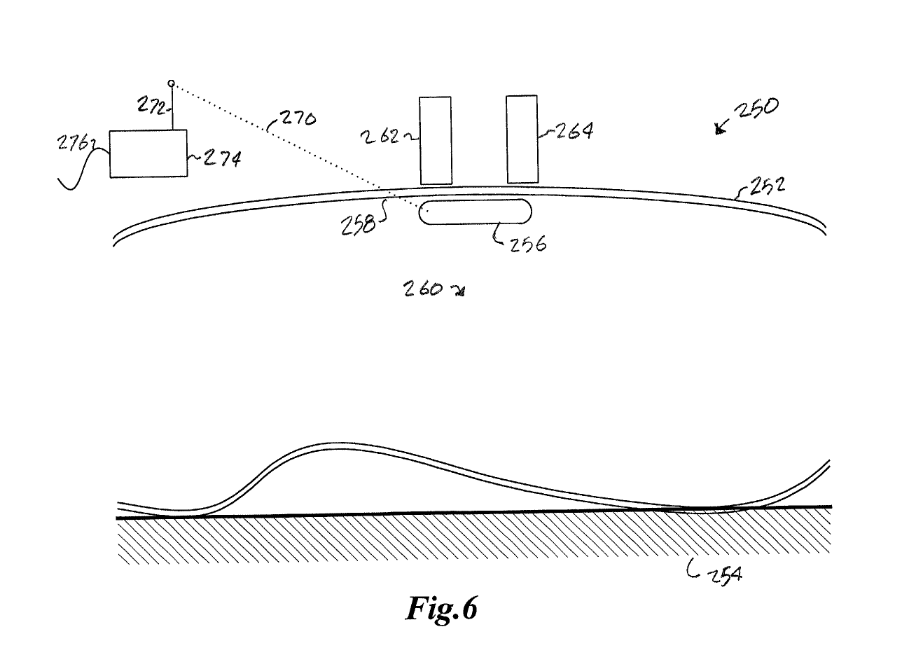

FIG. 6 is a lengthwise section view depicting a laparoscopic surgery apparatus 250 in accordance with certain embodiments of the present invention. Laparoscopic surgery apparatus 250 incorporates a surface 254 for supporting the patient 252, one or more laparoscopic instruments 256, and magnetic sources 262 and 264. FIG. 6, laparoscopic instrument 256 is shown disposed within the outer surface 258 of the patient 252, such that the laparoscopic instrument 256 is disposed within an inner cavity 260 within the patient 252.

The system may be constructed using, e.g., a high-resolution charge-coupled device (CCD) camera or even an analog camera. The system may even include a raster system. Software may be used to facilitate automated single-scan capture and analysis of images captured with a CCD camera. The sensitivity, reliability and simplicity of operation of the system may be evaluated by direct comparison to conventional images captured using conventional laparoscopic instruments. Other image capture systems may be used in conjunction with the imaging system. For example, fiber optic leads may be placed close to the image and the image transferred for capture outside the body. In addition, wavelengths outside visible light may be captured by the imaging system.

Owing to the use of magnetic fields to position, orient, and affix laparoscopic instrument 256 within the body cavity 260, a surgeon's control over the position and orientation of laparoscopic instrument 256 can be controlled with much greater flexibility. As can be seen in FIG. 6, the position and orientation of laparoscopic instrument 256 is controlled in part by magnetic field sources 262 and 264. The laparoscopic instrument 256 shown in FIG. 6 is fixed in place by the manipulation, by magnetic field sources 262 and 264, of one or more magnetically attractive portions of the laparoscopic instrument 256.

In contrast to the embodiments shown in FIGS. 1-5, laparoscopic surgery apparatus 250 incorporates a laparoscopic instrument 256 having no direct physical connection to the world outside the body of the patient 252. In this embodiment, all positional and orientation control of laparoscopic instrument 256 must be effectuated through the use of magnetic field generators 262 and 264.

In certain embodiments, a certain amount of control input may be provided to laparoscopic instrument 256 through wireless link 270, which communicates to wireless transmitter/receiver 274 through antenna 272. In embodiments in which laparoscopic instrument 256 incorporates an endoscope or other type of sensor, wireless link 270 may be used to relay video or other sensory information from laparoscopic instrument 256 to the outside world. Information transmitted and received through wireless link 270 is relayed to other portions of the laparoscopic apparatus (not shown) via link 276.

A wide variety of permanent magnets may be used with the present invention, such as rare earth magnets, ceramic magnets, alnico magnets, which may be rigid, semi-rigid or flexible. Flexible magnets are made by impregnating a flexible material such as neoprene rubber, vinyl, nitrile, nylon or a plastic with a material such as iron having magnetic characteristics. Other examples of magnets for use as described hereinabove, are rare earth magnets include neodymium iron boron (NdFeB) and Samarium Cobalt (SmCo) classes of magnets. Within each of these classes are a number of different grades that have a wide range of properties and application requirements. Rare earth magnets are available in sintered as well as in bonded form.

Ceramic magnets are sintered permanent magnets composed of Barium Ferrite (BaO (Fe2O3)n) or Strontium Ferrite (SnO (Fe2O3)n), where n is a variable quantity of ferrite. Also known as anisotropic hexaferrites, this class of magnets is useful due to its good resistance to demagnetization and its low cost. While ceramic magnets tend to be hard and brittle, requiring special machining techniques, these magnets can be used in magnetic holding devices having very precise specifications. Anisotropic grades are oriented during manufacturing, and must be magnetized in a specified direction. Ceramic magnets may also be isotropic, and are often more convenient due to their lower cost. Ceramic magnets are useful in a wide range of applications and can be pre-capped or formed for use with the present invention.

The embodiments and examples set forth herein are presented to best explain the present invention and its practical application and to thereby enable those skilled in the art to make and utilize the invention. Those skilled in the art, however, will recognize that the foregoing description and examples have been presented for the purpose of illustration and example only. Other variations and modifications of the present invention will be apparent to those of skill in the art, and it is the intent of the appended claims that such variations and modifications be covered. The description as set forth is not intended to be exhaustive or to limit the scope of the invention. Many modifications and variations are possible in light of the above teaching without departing from the spirit and scope of the following claims. It is contemplated that the use of the present invention can involve components having different characteristics. It is intended that the scope of the present invention be defined by the claims appended hereto, giving full cognizance to equivalents in all respects.

What is claimed is:

1. A method of reorienting a laparoscopic instrument having a magnetically attractive element with at least one principal magnetic axis from a first orientation to a second orientation, the method comprising the steps of:
generating a magnetic field in alignment with the principal magnetic axis of the magnetically-attractive element; and
reorienting the magnetic field in such a manner as to reorient the laparoscopic instrument to the second orientation.

2. The method of claim 1 wherein the magnetically attractive element is a piece of ferromagnetic material.

3. The method of claim 2 wherein the ferromagnetic material is iron.

4. The method of claim 1 wherein the laparoscopic instrument is a camera.

5. The method of claim 1 wherein some portion of the magnetic field is generated by a permanent magnet.

6. The method of claim 1 wherein some portion of the magnetic field is generated by an electromagnet.

7. The method of claim 6 wherein the magnetic field is time-varying.

8. A method of reorienting a laparoscopic instrument from a first orientation to a second orientation, the method comprising the steps of:
rigidly attaching to the laparoscopic instrument a magnetically-attractive element having at least one principal magnetic axis;
generating a magnetic field in alignment with the principal magnetic axis of the magnetically-attractive element; and
reorienting the magnetic field in such a manner as to reorient the laparoscopic instrument to the second orientation.

9. The method of claim 8 wherein the magnetically-attractive element is a piece of ferromagnetic material.

10. The method of claim 9 wherein the ferromagnetic material is iron.

11. The method of claim 8 wherein the laparoscopic instrument is a camera.

12. The method of claim 8 wherein some portion of the magnetic field is generated by a permanent magnet.

13. The method of claim 8 wherein some portion of the magnetic field is generated by an electromagnet.

14. The method of claim 13 wherein the magnetic field is time-varying.

15. A method of capturing an image from within a human body comprising the steps of:
inserting a instrument having light-sensitive elements and a magnetically-attractive element into the body;
applying a magnetic field to the magnetically-attractive element in such a manner as to orient the instrument to a desired orientation; and
exposing the light-sensitive elements of the instrument to light reflected from a desired internal feature.

16. The method of claim 15 wherein the magnetically attractive element is a piece of ferromagnetic material.

17. The method of claim 16 wherein the ferromagnetic material is iron.

18. The method of claim 15 wherein the laparoscopic instrument is a camera.

19. A method of repositioning a laparoscopic instrument having a magnetically attractive element with at least one principal magnetic axis from a first position to a second position, the method comprising the steps of:
applying to the magnetically-attractive element a first magnetic field aligned with a first principal axis; and
applying to the magnetically attractive element a second magnetic field aligned with a second principal axis.

20. A method of moving a laparoscopic instrument having a magnetically-attractive element from a first orientation and second position to a second orientation and second position, the method comprising the steps of:
detecting the first position;
applying to the magnetically attractive element a first magnetic field aligned with a first principal axis so as to translate the laparoscopic instrument from the first position to the second position;
detecting the laparoscopic instrument orientation; and
applying to the magnetically attractive element a second magnetic field so as to reorient the laparoscopic instrument to the second orientation.
